SEQUENCE LISTING (16 sequences)

SEQ ID NO: 1 - DNA, 3330 bp, Gallus gallus
catatgcaccaccaccaccaccacctattaaagagtgcagaatcagaaaaggagatggcgaacatgaaagaggaattcgagaagaccaaagaggaactggcgaaaagcgaagcgaagcgtaaagagctggaggaaaagatggtggttctgctgcaggagaagaacgatctgcagctgcaagttcaagcggaagcggacagcctggcggatgcggaggaacgttgcgaccaactgatcaagaccaaaattcagctggaggcgaagatcaaagaggttaccgaacgtgcggaggacgaggaagagattaacgcggaactgaccgcgaagaaacgtaaactggaagatgagtgcagcgaactgaagaaagacatcgacgatctggagctgaccctggcgaaggtggaaaaggagaaacacgcgaccgagaacaaggtgaaaaacctgaccgaagagatggcggttctggatgaaaccattgcgaaactgaccaaggaaaagaaagcgctgcaggaagcgcaccagcaaaccctggacgatctgcaagtggaagaggacaaagttaacaccctgaccaaggcgaaaaccaagctggaacagcaagttgacgatctggagggtagcctggaacaggagaagaaactgcgtatggatctggaacgtgcgaagcgtaagctggagggcgacctgaagctggcgcacgacagcatcatggatctggagaacgacaagcagcaactggatgaaaaactgaagaaaaaggacttcgagatcagccaaattcagagcaaaattgaagacgagcaagcgctgggtatgcaactgcagaaaaagattaaggagctgcaggcgcgtatcgaagagctggaagaggaaattgaagcggagcgtaccagccgtgcgaaagcggaaaagcaccgtgcggatctgagccgtgaactggaggaaatcagcgagcgtctggaggaagcgggtggcgcgaccgcggcgcagattgagatgaacaaaaagcgtgaagcggagtttcagaaaatgcgtcgtgacctggaggaagcgaccctgcagcatgaggcgaccgcggcggcgctgcgtaaaaagcatgcggatagcaccgcggaactgggcgagcagatcgacaacctgcaacgtgtgaaacagaagctggaaaaagagaagagcgaactgaaaatggagattgacgatctggcgagcaacatggaaagcgttagcaaagcgaaagcgaacctggagaagatgtgccgtaccctggaagatcagctgagcgagatcaaaaccaaagaggagcaaaaccagcgtatgattaacgacctgaacacccaacgtgcgcgtctgcagaccgaaaccggcgagtacagccgtcaggcggaggaaaaggatgcgctgatcagccaactgagccgtggtaaacagggctttacccagcaaatcgaggaactgaagcgtcacctggaggaagagattaaagcgaagaacgcgctggcgcatgcgctgcaaagcgcgcgtcacgactgcgaactgctgcgtgagcaatacgaagaggaacaggaagcgaaaggtgaactgcagcgtgcgctgagcaaagcgaacagcgaagtggcgcagtggcgtaccaaatatgagaccgatgcgatccaacgtaccgaggaactggaggaagcgaaaaagaaactggcgcaacgtctgcaggacgcggaggaacacgtggaagcggttaacgcgaagtgcgcgagcctggagaaaaccaagcaacgtctgcagaacgaagttgaggatctgatggtggacgttgaacgtagcaacgcggcgtgcgcggcgctggataagaaacagaagaacttcgacaagatcctggcggaatggaaacaaaagtacgaggaaacccagaccgaactggaggcgagccagaaggaaagccgtagcctgagcaccgagctgtttaaaatgaagaacgcgtatgaggaaagcctggatcacctggaaaccctgaaacgtgagaacaagaacctgcagcaagagatcgcggacctgaccgaacagattgcggagggtggcaaggcggtgcacgaactggagaaagtgaagaaacacgttgaacaagagaaaagcgagctgcaggcgagcctggaggaagcggaagcgagcctggagcacgaggaaggtaaaatcctgcgtctgcaactggaactgaaccagatcaagagcgagattgaccgtaaaatcgcggaaaaggatgaggaaatcgaccaactgaagcgtaaccacctgcgtattgttgagagcatgcagagcaccctggatgcggaaatccgtagccgtaacgaggcgctgcgtctgaagaaaaagatggagggtgacctgaacgaaatggagattcagctgagccatgcgaaccgtatggcggcggaagcgcaaaagaacctgcgtaacacccagggcaccctgaaagatacccaaatccacctggacgatgcgctgcgtacccaagaagacctgaaagagcaggtggcgatggttgaacgtcgtgcgaacctgctgcaggcggaagtggaggaactgcgtggcgcgctggaacagaccgagcgtagccgtaaggtggcggaacaagagctgctggacgcgaccgagcgtgttcaactgctgcacacccagaacaccagcctgattaacaccaaaaagaaactggagaccgatatcgttcaaattcagagcgaaatggaggacaccatccaggaagcgcgtaacgcggaggagaaagcgaagaaagcgattaccgatgcggcgatgatggcggaggaactgaagaaagaacaggacaccagcgcgcacctggagcgtatgaagaaaaacatggatcaaaccgtgaaggacctgcacgttcgtctggatgaagcggagcagctggcgctgaagggtggcaagaaacaactgcagaaactggaggcgcgtgtgcgtgaactggagggtgaagttgacagcgaacaaaagcgtagcgcggaggcggtgaaaggcgttcgtaagtacgaacgtcgtgtgaaagagctgacctatcagtgcgaggaagatcgtaagaacatcctgcgtctgcaggacctggtggataagctgcaaatgaaagttaagagctataaacgtcaggcggaggaagcggaggaactgagcaacgttaacctgagcaaattccgtaagatccaacacgaactggaggaagcggaggaacgtgcggacattgcggagagccaggtgaacaaactgcgtgttaagagccgtgaaattcacggtaagaaaatcgaggaagaggaataatgaaagctt

SEQ ID NO: 2 - DNA, 1164 bp, Gallus gallus
catatgcaccaccaccaccaccactgtgatgaagatgaaacaactgctttagtttgcgacaacggtagcggtctggtgaaagcgggttttgcgggtgatgatgcgccgcgtgcggtgtttccgagcatcgttggtcgtccgcgtcaccagggcgtgatggttggtatgggccaaaaagacagctacgttggtgatgaggcgcagagcaagcgtggcatcctgaccctgaaatatccgattgaacacggtatcattaccaactgggacgatatggagaagatctggcaccacaccttctacaacgaactgcgtgttgcgccggaggaacatccgaccctgctgaccgaggcgccgctgaacccgaaggcgaaccgtgagaaaatgacccagatcatgttcgaaacctttaacgtgccggcgatgtacgttgcgattcaagcggtgctgagcctgtatgcgagcggtcgtaccaccggcatcgttctggacagcggtgatggcgtgacccacaacgttccgatctacgaaggttatgcgctgccgcacgcgattatgcgtctggacctggcgggtcgtgacctgaccgattacctgatgaagattctgaccgagcgtggttatagcttcgttaccaccgcggagcgtgaaatcgtgcgtgacattaaggaaaaactgtgctacgttgcgctggattttgagaacgaaatggcgaccgcggcgagcagcagcagcctggagaaaagctatgaactgccggatggtcaggtgatcaccattggcaacgagcgttttcgttgcccggaaaccctgttccaaccgagctttatcggtatggagagcgcgggcattcacgaaaccacctacaacagcatcatgaagtgcgacatcgatattcgtaaagacctgtacgcgaacaacgtgatgagcggtggcaccaccatgtatccgggtatcgcggatcgtatgcagaaggagattaccgcgctggcgccgagcaccatgaagattaaaatcattgctccgccggaacgtaaatacagcgtttggatcggtggcagcattctggcgagcctgagcaccttccagcaaatgtggatcaccaagcaagagtatgatgaagcgggcccgagcattgtgcaccgtaaatgcttttaatgaaagctt

SEQ ID NO: 3 - protein, 1105 aa, Gallus gallus
MHHHHHHLLKSAESEKEMANMKEEFEKTKEELAKSEAKRKELEEKMVVLLQEKNDLQLQVQAEADSLADAEERCDQLIKTKIQLEAKIKEVTERAEDEEEINAELTAKKRKLEDECSELKKDIDDLELTLAKVEKEKHATENKVKNLTEEMAVLDETIAKLTKEKKALQEAHQQTLDDLQVEEDKVNTLTKAKTKLEQQVDDLEGSLEQEKKLRMDLERAKRKLEGDLKLAHDSIMDLENDKQQLDEKLKKKDFEISQIQSKIEDEQALGMQLQKKIKELQARIEELEEEIEAERTSRAKAEKHRADLSRELEEISERLEEAGGATAAQIEMNKKREAEFQKMRRDLEEATLQHEATAAALRKKHADSTAELGEQIDNLQRVKQKLEKEKSELKMEIDDLASNMESVSKAKANLEKMCRTLEDQLSEIKTKEEQNQRMINDLNTQRARLQTETGEYSRQAEEKDALISQLSRGKQGFTQQIEELKRHLEEEIKAKNALAHALQSARHDCELLREQYEEEQEAKGELQRALSKANSEVAQWRTKYETDAIQRTEELEEAKKKLAQRLQDAEEHVEAVNAKCASLEKTKQRLQNEVEDLMVDVERSNAACAALDKKQKNFDKILAEWKQKYEETQTELEASQKESRSLSTELFKMKNAYEESLDHLETLKRENKNLQQEIADLTEQIAEGGKAVHELEKVKKHVEQEKSELQASLEEAEASLEHEEGKILRLQLELNQIKSEIDRKIAEKDEEIDQLKRNHLRIVESMQSTLDAEIRSRNEALRLKKKMEGDLNEMEIQLSHANRMAAEAQKNLRNTQGTLKDTQIHLDDALRTQEDLKEQVAMVERRANLLQAEVEELRGALEQTERSRKVAEQELLDATERVQLLHTQNTSLINTKKKLETDIVQIQSEMEDTIQEARNAEEKAKKAITDAAMMAEELKKEQDTSAHLERMKKNMDQTVKDLHVRLDEAEQLALKGGKKQLQKLEARVRELEGEVDSEQKRSAEAVKGVRKYERRVKELTYQCEEDRKNILRLQDLVDKLQMKVKSYKRQAEEAEELSNVNLSKFRKIQHELEEAEERADIAESQVNKLRVKSREIHGKKIEEEE

SEQ ID NO: 4 - protein, 383 aa, Gallus gallus
MHHHHHHCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 5 - protein, 1938 aa, Bos taurus
MSSDQEMAVFGEAAPYLRKSEKERIEAQNKPFDAKTSVFVADPKESFVKATVQSREGGKVTAKTEAGATVTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAYVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVEQVYNAVGALAKAVYDKMFLWMVARINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDMSFKNKLYEQHLGKSNNFQKPKPAKGKAEAHFSLIHYAGTVDYNITGWLDKNKDPLNETVVGLYQKSSVKTLALLFSGPASGEAEGGPKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLASIDVDHTQYKFGHTKVFFKAGLLGLLEEMRDEKLAQLITRTQARCRGFLARVEYQKMVERRESIFCIQYNVRAFMNVKHWPWMKLYFKIKPLLKSAETEKEMANMKEEFEKTKEELAKSEAKRKELEEKMVTLTQEKNDLQLQVQSEADALADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSEMKMEIDDLASNMETVSKAKGNLEKMCRALEDQLSELKTKEDEQQRLINDLTTQRARLQTESGEFSRQLDEKDALVSQLSRGKQAFTQQIEELKRQLEEEIKAKSALAHALQSARHDCDLLREQYEEEQEGKAELQRAMSKANSEVAQWRTKYETDAIQRTEELEEAKKKLAQRLQDAEEHVEAVNAKCASLEKTKQRLQNEVEDLMIDVERTNAACAALDKKQRNFDKILSEWKQKYEETHAELEASQKESRSLSTELFKIKNAYEESLDQLETLKRENKNLQQEISDLTEQIAEGGKRIHELEKVKKQVEQEKSEIQAALEEAEASLEHEEGKILRIQLELNQVKSEIDRKIAEKDEEIDQLKRNHIRIVESMQSTLDAEIRSRNDAIRLKKKMEGDLNEMEIQLNHANRMAAEALKNYRSTQAILKDTQIHLDDALRGQEDLKEQLAMVERRANLLQAEIEELRATLEQTERSRKIAEQELLDASERVQLLHTQNTSLINTKKKLETDITQIQGEMEDIIQEARNAEEKAKKAITDAAMMAEELKKEQDTSAHLERMKKNLEQTVKDLQHRLDEAEQLALKGGKKQIQKLEARVRELEGEVESEQKRNVEAVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 6 - protein, 1938 aa, Bos taurus
MSSDQEMAIFGEAAPYLRKSEKERIEAQNKPFDAKTSVFVADPKESFVKATVQSREGGKVTAKTEAGATVTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAYVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVEQVYNAVGALAKAVYDKMFLWMVARINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDMSFKNKLYEQHLGKSNNFQKPKPAKGKAEAHFSLIHYAGTVDYNITGWLDKNKDPLNETVVGLYQKSSVKTLALLFSGPASGEAEGGPKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLASIDVDHTQYKFGHTKVFFKAGLLGLLEEMRDEKLAQLITRTQARCRGFLARVEYQKMVERRESIFCIQYNVRAFMNVKHWPWMKLYFKIKPLLKSAETEKEMANMKEEFEKTKEELAKSEAKRKELEEKMVTLTQEKNDLQLQVQSEADALADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSEMKMEIDDLASNMETVSKAKGNLEKMCRALEDQLSELKTKEDEQQRLINDLTTQRARLQTESGEFSRQLDEKDALVSQLSRGKQAFTQQIEELKRQLEEEIKAKSALAHALQSARHDCDLLREQYEEEQEGKAELQRAMSKANSEVAQWRTKYETDAIQRTEELEEAKKKLAQRLQDAEEHVEAVNAKCASLEKTKQRLQNEVEDLMIDVERTNAACAALDKKQRNFDKILSEWKQKYEETHAELEASQKESRSLSTELFKIKNAYEESLDQLETLKRENKNLQQEISDLTEQIAEGGKRIHELEKVKKQVEQEKSEIQAALEEAEASLEHEEGKILRIQLELNQVKSEIDRKIAEKDEEIDQLKRNHIRVVESMQSTLDAEIRSRNDAIRLKKKMEGDLNEMEIQLNHANRMAAEALKNYRNTQAILKDTQIHLDDALRGQEDLKEQLAMVERRANLLQAEIEELRATLEQTERSRKIAEQELLDASERVQLLHTQNTSLINTKKKLETDITQIQGEMEDIIQEARNAEEKAKKAITDAAMMAEELKKEQDTSAHLERMKKNLEQTVKDLQHRLDEAEQLALKGGKKQIQKLEARVRELEGEVESEQKRNVEAVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 7 - protein, 1939 aa, Sus scrofa
MSSDQEMAIFGEAAPYLRKSEKERIEAQNKPFDAKTSVFVAEPKESFVKGTVQSREGGKVTVKTEAGATLTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVQQVYNAVGALAKAVYDKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKVEAHFSLIHYAGTVDYNITGWLDKNKDPLNETVVGLYQKSSVKTLAFLFTGAAGADAEAGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGLLEEMRDEKLAQLITRTQARCRGFLARVEYQKMVERRESIFCIQYNIRAFMNVKHWPWMKLYFKIKPLLKSAETEKEMANMKEEFEKTKESLAKAEAKRKELEEKMVALMQEKNDLQLQVQAEADSLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAATLRKKHADSVAELGEQIDNLQRVKQKLEKEKSEMKMEIDDLASNMETVSKAKGNLEKMCRTLEDQLSELKTKEEEQQRLINDLTAQRARLQTESGEYSRQLDEKDTLVSQLSRGKQAFTQQIEELKRQLEEEIKAKSALAHAVQSSRHDCDLLREQYEEEQEAKAELQRAMSKANSEVAQWRTKYETDAIQRTEELEEAKKKLAQRLQDAEEHVEAVNAKCASLEKTKQRLQNEVEDLMIDVERSNAACAALDKKQRNFDKILAEWKQKYEETHAELEASQKESRSLSTELFKVKNAYEESLDQLETLKRENKNLQQEISDLTEQIAEGGKRIHELEKIKKQVEQEKSEIQAALEEAEASLEHEEGKILRIQLELNQVKSEVDRKIAEKDEEIDQLKRNHVRVVESMQSMLDAEIRSRNDAIRLKKKMEGDLNEMEIQLNHANRMAAEALRNYRNTQGILKDTQIHLDDALRSQEDLKEQLAMVERRANLLQAEIEELRATLEQTERSRKVAEQELLDASERVQLLHTQNTSLINTKKKLETDISQIQGEMEDIIQEARNAEEKAKKAITDAAMMAEELKKEQDTSAHLERMKKNLEQTVKDLQHRLDEAEQLALKGGKKQIQKLEARVRELEGEVESEQKRNVETVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 8 - protein, 1939 aa, Ovis aries
MSSDQEMAIFGEAAPYLRKSEKERIEAQNKPFDAKTSVFVADPKESFVKATVQSREGGKVTAKTEAGATVTVKEDQVFPMNPPKFDKIEDMAMMTHLHEPGVLYNLKERYAAWMIYTYSGLFCVTVNPYKWLPVYNAEVVAAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVEQVYNAVGALAKAIYDKMFLWMVTRINQQLDTKQPRQYFIGVLDIAGFEIFDFNSFEQLCINFTNEKLQQFFNHHMFVLEQEEYKREGIEWEFIDFGMDLAACIELIEKPMGIFSILEEECMFPKATDTSFKNKLYEQHLGKSNNFQKPKPAKGKAEAHFSLIHYAGTVDYNITGWLDKNKDPLNETVVGLYQKSSVKTLAFLFSGAASAEAEGGGAKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDEKLAQLITRTQARCRGFLARVEYQRMVERRESIFCIQYNVRAFMNVKHWPWMKLYFKIKPLLKSAETEKEMANMKEEFEKTKEELAKSEAKRKELEEKMVTLTQEKNDLQLQVQSEADALADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALGMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQLDNLQRVKQKLEKEKSEMKMEIDDLASNMETVSKAKGNLEKMCRTLEDQLSELKTKEDEQQRLINDLTTQRARLQTESGEFSRQLDEKDALVSQLSRGKQAFTQQIEELKRQLEEEIKAKSALAHALQSARHDCDLLREQYEEEQEGKAELQRAMSKANSEVAQWRTKYETDAIQRTEELEEAKKKLAQRLQDAEEHVEAVNAKCASLEKTKQRLQNEVEDLMIDVERTNAACAALDKKQRNFDKILSEWKQKYEETHAELEASQKESRSLSTELFKIKNAYEESLDQLETLKRENKNLQQEISDLTEQIAEGGKRIHELEKVKKQVEQEKSEIQAALEEAEASLEHEEGKILRIQLELNQVKSEIDRKIAEKDEEIDQLKRNHIRVVESMQSTLDAEIRSRNDAIRLKKKMEGDLNEMEIQLNHANRMAAEALKNYRNTQAILKDTQLHLDDALRGQEDLKEQLAMVERRANLLQAEIEELRATLEQTERSRKIAEQELLDASERVQLLHTQNTSLINTKKKLETDITQIQGEMEDIIQEARNAEEKAKKAITDAAMMAEELKKEQDTSAHLERMKKNLEQTVKDLQHRLDEAEQLALKGGKKQIQKLEARVRELEAEVESEQKRNVETVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 9 - protein, 1939 aa, Capra hircus
MSSDQEMAIFGEAAPYLRKSEKERIEAQNKPFDAKTSVFVADPKESFVKATVQSREGGKVTAKTEAGATVTVKEDQVFPMNPPKFDKIEDMAMMTHLHEPGVLYNLKERYAAWMIYTYSGLFCVTVNPYKWLPVYNAEVVAAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVEQVYNAVGALAKAVYDKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLIHYAGTVDYNITGWLDKNKDPLNETVVGLYQKSSVKTLAFLFSGAASAEAEGGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDEKLAQLITRTQARCRGFLARVEYQRMVERRESIFCIQYNVRAFMNVKHWPWMKLYFKIKPLLKSAETEKEMANMKEEFEKTKEELAKSEAKRKELEEKMVTLTQEKNDLQLQVQSEADALADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALGMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQLDNLQRVKQKLEKEKSEMKMEIDDLASNMETVSKAKGNLEKMCRTLEDQLSELKTKEDEQQRLINDLTTQRARLQTESGEFSRQLDEKDALVSQLSRGKQAFTQQIEELKRQLEEEIKAKSALAHALQSARHDCDLLREQYEEEQEGKAELQRAMSKANSEVAQWRTKYETDAIQRTEELEEAKKKLAQRLQEAEEHVEAVNAKCASLEKTKQRLQNEVEDLMIDVERTNAACAALDKKQRNFDKILSEWKQKYEETHAELEASQKESRSLSTELFKIKNAYEESLDQLETLKRENKNLQQEISDLTEQIAEGGKRIHELEKVKKQVEQEKSEIQAALEEAEASLEHEEGKILRIQLELNQVKSEIDRKIAEKDEEIDQLKRNHIRVVESMQSTLDAEIRSRNDAIRLKKKMEGDLNEMEIQLNHANRMAAEALKNYRNTQAILKDTQLHLDDALRGQEDLKEQLAMVERRANLLQAEIEELRATLEQTERSRKIAEQELLDASERVQLLHTQNTSLINTKKKLETDITQIQGEMEDIIQEARNAEEKAKKAITDAAMMAEELKKEQDTSAHLERMKKNLEQTVKDLQHRLDEAEQLALKGGKKQIQKLEARVRELEAEVESEQKRNVETVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 10 - protein, 1940 aa, Anas platyrhynchos
MSSDSEMAIFGEAAPYLRKSEKERIEAQNKPFDAKSSVFVVHPKESFVKGTIQSKESGKVTVKTEGGETLTVKEDQIFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGEKKKEEQPGKMQGTLEDQIISANPLLEAFGNAKTVRNDNSSRFGKFIRIHFGATGKLASADIETYLLEKSRVTFQLKAERSYHIFYQIMSNKKPELIDMLLITTNPYDYQFVSQGEITVPSINDQEELIATDSAIDILGFTADEKTAIYKLTGAVMHYGNLKFKQKQREEQAEPDGTEVADKAAYLMGLNSADLLKALCYPRVKVGNEYVTKGQTVQQVNNAVGALAKAVYEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSNNFQKPKPGKGKAEAHFSLVHYAGTVDYNISGWLEKNKDPLNETVIGLYQKSSVKTLALLFASAGGEAEASGGGGKKGGKKKGSSFQTVSALFRENLNKLMTNLRSTHPHFVRCIIPNETKTPGAMEHELVLHQLRCNGVLEGIRICRKGFPSRVLYADFKQRYKVLNASAIPEGQFIDSKKASEKLLGSIDVDHTQYKFGHTKVFFKAGLLGLLEEMRDEKLAQLITRTQARCRGFLMRVEYQRMVERRESIFCIQYNVRAFMNVKHWPWMKLFFKIKPLLKSAESEKEMANMKGEFEKTKEELAKSEAKRKELEEKMVSLLQEKNDLQLQVQAEADSLADAEERCDQLIKTKIQLEAKIKEVTERAEDEEEINAELTAKKRKLEDECSELKKDIDDLELTLAKVEKEKHATENKVKNLTEEMASLDETIAKLTKEKKALQEAHQQTLDDLQAEEDKVNTLTKAKTKLEQQVDDLEGSLEQEKKLRMDLERAKRKLEGDLKLAHDSIMDLENDKQQLDEKLKKKDFEISQIQSKIEDEQALGMQLQKKIKELQARIEELEEEIEAERTSRAKAEKHRADLSRELEEISERLEEAGGATAAQIEMNKKRESEFQKMRRDLEEATLQHEATAAALRKKHADSTAELGEQIDNLQRVKQKLEKEKSELKMEIDDLASNMESVSKAKANLEKMCRTLEDQLSEVKTKEEEHQRMVNDLNAQRARLQTESGEYSRQVEEKDALISQLSRGKQAFTQQIEELKRHLEEEIKAKSALAHALQSARHDCDLLREQYEEEQEAKGELQRALSKANSEVAQWRTKYETDAIQRTEELEEAKKKLAQRLQDAEEHVEAVNAKCASLEKTKQRLQNEVEDLMIDVERSNAACAALDKKQKNFDKILAEWKQKYEETQAELEASQKESRSLSTELFKMKNAYEESLDHLETLKRENKNLQQEISDLTEQIAEGGKAIHELEKVKKQIEQEKSEIQAALEEAEASLEHEEGKILRLQLELNQVKSEIDRKIAEKDEEIDQLKRNHLRIVESMQSTLDAEIRSRNEALRLKKKMEGDLNEMEIQLSHANRQAAEAQKNLRNTQGVLKDTQIHLDDALRTQEDLKEQVAMVERRANLLQAEIEELRAALEQTERSRKVAEQELMDASERVQLLHTQNTSLINTKKKLETDIAQIQGEMEDTIQEARNAEEKAKKAITDAAMMAEELKKEQDTSAHLERMKKNLDQTVKDLQHRLDEAEQLALKGGKKQIQKLEARVRELEGEVDAEQKRSAEAVKGVRKYERRVKELTYQSEEDRKNILRLQDLVDKLQMKVKSYKRQAEEAEELSNVNLSKFRKIQHELEEAEERADIAESQVNKLRVKSREFHSKKIEEEE

SEQ ID NO: 11 - protein, 1939 aa, Oryctolagus cuniculus
MSSDADMAVFGEAAPYLRKSEKERIEAQNKPFDAKNSVFVVDPKESYVKSTVQSREGGKVTVKTEAGATVTVKEDQVFSMNPPKYDKIEDMAMMTHLHEPAVLYNLKERYAAWMIYTYSGLFCVTVNPYKWLPVYNPEVVTAYRGKKRQEAPPHIFSISDNAYQFMLTDRENQSILITGESGAGKTVNTKRVIQYFATIAITGDKKKEEPTPGKMQGTLEDQIISANPLLEAFGNAKTVRNDNSSRFGKFIRIHFGATGKLASADIETYLLEKSRVTFQLKAERSYHIFYQIMSNKKPELIEMLLITTNPYDFAFVSQGEITVPSIDDQEELMATDSAVDILGFTSDEKVAIYKLTGAVMHYGNLKFKQKQREEQAEPDGTEVADKAAYLTSLNSADLLKALCYPRVKVGNEYVTKGQTVQQVYNAVGALAKAIYEKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLVHYAGTVDYNITGWLDKNKDPLNETVVGLYQKSGMKTLAFLFSGAQTGEEGGGGGKKGGKKKGSSFQTVSALFRENLNKLMTNLRSTHPHFVRCLIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDEKLAQLITRTQAICRGYLTRVEFKKMMERRESIFCIQYNIRAFMNVKHWPWMKLYFKIKPLLKSAETEKEMANMKEEFEKTKEELAKSEAKRKELEEKMVALMQEKNDLQLQVQAEADGLADAEERCDQLIKTKIQLEAKIKEVTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMTNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSELKMEIDDLAGNMETVSKAKGNLEKMCRTLEDQLSEVKTKEEEHQRLINELSAQKARLHTESGEFSRQLDEKDAMVSQLSRGKQAFTQQIEELKRQLEEETKAKSALAHALQSSRHDCDLLREQYEEEQEAKAELQRAMSKANSEVSQWRTKYETDAIQRTEELEEAKKKLAQRLQDAEEHVEAVNSKCASLEKTKQRLQNEVEDLMIDVERSNAACAALDKKQRNFDKVLAEWKHKYEETQAELEASQKESRSLSTEVFKVKNAYEESLDHLETLKRENKNLQQEISDLTEQIAESGKHIHELEKVKKQIDQEKSELQAALEEAEGSLEHEEGKILRIQLELNQVKSEIDRKIAEKDEEIDQLKRNHLRVVESMQSTLDAEIRSRNDALRIKKKMEGDLNEMEIQLNHANRQAAEAIKNLRNTQGILKDTQLHLDDAVRGQDDLKEQLAMVERRANLMQAEIEELRASLEQTERSRRVAEQELLDASERVQLLHTQNTSLINTKKKLETDISQIQGEMEDIVQEARNAEEKAKKAITDAAMMAEELKKEQDTSAHLERMKKNMEQTVKDLQHRLDEAEQLALKGGKKQIQKLEARVRELENEVESEQKRNVEAVKGLRKHERRVKELTYQTEEDRKNVLRLQDLVDKLQSKVKAYKRQAEEAEEQSNINLSKFRKLQHELEEAEERADIAESQVNKLRVKSRDVHSKVISEE

SEQ ID NO: 12 - protein, 377 aa, Bos taurus
MCDEDETTALVCDNGSGLVKAGFAGDDAPRAVFPSIVGRPRHQGVMVGMGQKDSYVGDEAQSKKGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 13 - protein, 465 aa, Sus scrofa
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKVGGGPRPCPLVGSRLRLRPPDRLSLSYRSSPRRSASTPCGSAAPSWPRCPPSSRCGSPSRSTTRPAHLSSTANASKPHSNSCDFSSGRRICSSAVADLEPPSGNCSPTACVAAAQTDTMFITCTYINLFTSFCYF

SEQ ID NO: 14 - DNA, 1175 bp, Gallus gallus
ctcgagaaaagaatgcaccatcatcatcatcactgcgacgaagacgaaacaactgccttagtatgcgataacggttcaggtttagtcaaagccggttttgctggtgatgatgctccaagagcagtttttccatctattgttggtagaccaagacatcaaggtgttatggttggtatgggtcaaaaagattcttatgttggtgatgaagcacaatcaaagagaggtatcttgactttgaagtacccaatcgaacatggtatcatcacaaactgggatgatatggaaaagatctggcatcatactttctacaacgaattaagagttgctccagaagaacatccaactttgttaacagaagctccattgaacccaaaggcaaacagagaaaagatgactcaaatcatgttcgaaacattcaacgttccagctatgtacgttgctattcaagcagttttgtctttatacgcatcaggtagaactacaggtattgttttagattcaggtgatggtgttactcataacgttccaatctacgaaggttacgctttgccacatgcaattatgagattggatttagctggtagagatttgactgattacttgatgaagatcttgacagaaagaggttactctttcgttactacagcagaaagagaaatcgttagagatatcaaggaaaagttgtgttatgttgctttggattttgaaaatgaaatggcaacagctgcatcttcatcttcattagaaaaatcatacgaattgccagatggtcaagttatcactatcggtaacgaaagattcagatgtccagaaacattgttccaaccatctttcatcggtatggaatcagctggtatccatgaaactacatacaactctatcatgaaatgtgatattgatattagaaaagatttatatgctaataatgttatgtcaggtggtactacaatgtacccaggtatcgcagatagaatgcaaaaggaaatcactgctttggcaccatctacaatgaagatcaagatcatcgctccaccagaaagaaaatactctgtttggattggtggttcaatcttggcatctttgtcaacattccaacaaatgtggattaccaagcaagaatacgacgaagcaggtccatcaatagttcacagaaagtgtttttaatgagcggccgc

SEQ ID NO: 15 - DNA, 3341 bp, Gallus gallus
ctcgagaaaagaatgcaccaccaccaccaccacttattgaaatctgctgaatccgaaaaggaaatggctaacatgaaggaagaatttgaaaagaccaaggaagaattagcaaagtcagaagctaagagaaaggaattggaagaaaagatggttgttttgttgcaagaaaagaatgatttgcaattacaagttcaagcagaagctgattctttagcagatgctgaagaaagatgtgatcaattgatcaagactaagatccaattagaagcaaagatcaaggaagttacagaaagagctgaagatgaagaagaaatcaacgcagaattgactgctaagaaaagaaagttagaagatgaatgttctgaattgaagaaagatatcgatgatttggaattaacattggcaaaggttgaaaaggaaaaacatgctactgaaaacaaggttaagaacttgacagaagaaatggcagttttggatgaaactatcgctaagttgacaaaggaaaagaaagctttgcaagaagctcatcaacaaactttggatgatttgcaagttgaagaagataaggttaacactttgacaaaggctaagacaaagttggaacaacaagttgatgatttggaaggttcattagaacaagaaaagaaattgagaatggatttagaaagagcaaagagaaagttagaaggtgatttaaagttggctcatgattctatcatggatttggaaaacgataaacaacaattggatgaaaagttgaaaaagaaagatttcgaaatctcacaaattcaatctaaaattgaagatgaacaagctttgggtatgcaattgcaaaagaaaattaaggaattacaagcaagaatcgaagaattggaagaagaaatcgaagctgaaagaacttcaagagcaaaagctgaaaaacatagagcagatttgtcaagagaattagaagaaatctctgaaagattagaagaagctggtggtgcaacagctgcacaaattgaaatgaacaagaaaagagaagctgaattccaaaagatgagaagagatttggaagaagcaactttacaacatgaagctacagctgcagctttgagaaagaaacatgcagattctactgctgaattaggtgaacaaatcgataacttgcaaagagttaagcaaaagttagaaaaggaaaaatcagaattgaaaatggaaatcgatgatttggcttctaacatggaatctgtttcaaaggcaaaggctaacttggaaaagatgtgtagaactttggaagatcaattatcagaaatcaagacaaaggaagaacaaaaccaaagaatgatcaacgatttgaacactcaaagagctagattgcaaactgaaacaggcgaatattctagacaagcagaagaaaaggatgctttgatctcacaattatctagaggtaaacaaggtttcacacaacaaatcgaagaattgaagagacatttagaagaagaaatcaaagcaaaaaatgcattggctcatgcattacaatctgctagacatgattgtgaattgttgagagaacaatacgaagaagaacaagaagctaagggtgaattgcaaagagcattatcaaaggctaactctgaagttgcacaatggagaactaaatacgaaacagatgctattcaaagaactgaagaattagaagaagctaagaaaaaattggctcaaagattgcaagatgctgaagaacatgttgaagctgttaacgcaaagtgtgcttcattggaaaagacaaagcaaagattgcaaaacgaagttgaagatttgatggttgatgttgaaagatctaatgcagcttgtgcagctttagataagaagcaaaagaatttcgataagatcttggcagaatggaagcaaaagtacgaagaaactcaaacagaattggaagcttcacaaaaagaatctagatcattgtctactgaattattcaagatgaagaacgcatacgaagaatctttggatcatttggaaactttgaagagagaaaataagaacttgcaacaagaaattgctgatttgacagaacaaattgcagaaggtggtaaagctgttcatgaattagaaaaagttaaaaaacatgttgaacaagaaaaatcagaattgcaagcttctttagaagaagctgaagcatcattggaacatgaagaaggcaagatcttgagattgcaattagaattgaatcaaattaaatctgaaattgatagaaaaattgctgaaaaagatgaagaaatcgatcaattgaagagaaaccatttgagaatcgttgaatcaatgcaatctacattggatgcagaaatcagatcaagaaacgaagctttgagattgaaaaagaaaatggagggtgatttaaacgaaatggaaatccaattgtctcatgcaaacagaatggcagctgaagctcaaaagaatttgagaaacactcaaggtacattgaaggatactcaaatccatttggatgatgcattgagaacacaagaagatttgaaggaacaagttgctatggttgaaagaagagcaaacttgttgcaagctgaagttgaagaattgagaggtgctttagaacaaactgaaagatctagaaaggttgcagaacaagaattgttagatgctacagaaagagttcaattgttacatactcaaaacacatcattgatcaacactaagaaaaaattggaaacagatatcgttcaaatccaatctgaaatggaagatactattcaagaagcaagaaatgctgaagaaaaagctaagaaagctattacagatgcagctatgatggcagaagaattgaagaaagaacaagacacttctgctcatttggaaagaatgaagaaaaatatggatcaaacagttaaggatttgcatgttagattggatgaagcagaacaattggctttgaaaggtggtaaaaaacaattgcaaaagttagaagctagagttagagaattagaaggtgaagttgattcagaacaaaagagatctgctgaagcagttaagggtgttagaaagtacgaaagaagagttaaggaattgacttaccaatgtgaagaagatagaaagaacatcttgagattgcaagatttggttgataaattacaaatgaaggttaagtcatacaagagacaagcagaagaagctgaagaattgtcaaacgttaacttgtctaagttcagaaagatccaacatgaattagaagaagctgaagaaagagcagatattgctgaatctcaagtaaataagttgagagtaaagtccagagaaatccacggtaaaaagatagaagaagaagaataatgagcggccgc

SEQ ID NO: 16 - protein, 1940 aa, Pecten maximus
MNIDYSDPDFQYLAVDRKKLMKEQTAAFDGKKNCWVPDEKEGFAPAEIQSSKGDEITVKITSDNSTRTVKKDDIQSMNPPKFEKLEDMANMTYLNEASVLHNLRSRYTSGLIYTYSGLFCIAVNPYRRLPIYTDSVISKYRGKRKTEIPPHLFSVADNAYQNMVTDRENQSCLITGESGAGKTENTKKVIMYLAKVACAVKKKTDEEEASDKKQGSLEDQIIQANPVLEAYGNAKTTRNNNSSRFGKFIRIHFGPTGKIAGADIETYLLEKSRVTYQQSAERNYHIFYQVCSNALPELNDIMLVTPDSGLYSFINQGCLTVDNIDDVEEFKLCDEAFDILGFTKEEKQSMFKCTASILHMGEMKFKQRPREEQAESDGTAEAEKVAFLCGINAGDLLKALLKPKVKVGTEMVTKGQNLNQVTNSVGALAKSLYDRMFNWLVKRVNKTLDTKAKRNYYIGVLDIAGFEIFDYNSFEQLCINYTNERLQQFFNHHMFILEQEEYKKEGIAWEFIDFGMDLQMCIDLIEKPMGILSILEEECMFPKADDKSFQDKLYQNHMGKNRMFTKPGKPTRPNQGPAHFELHHYAGNVPYSITGWLDKNKDPINENVVSLLSVSKEPLVAELFRAPEEPVGGGGKKKKGKSSAFQTISAVHRESLNKLMKNLYSTHPSFVRCIIPNELKQPGLVDAELVLHQLQCNGVLEGIRICRKGFPSRLIYSEFKQRYSILAPNAIPQGFVDGKTVSEKILTGLQMDPAEYRLGTTKVFFKAGVLGNLEEMRDERLSKIISMFQAHIRGYLIRKAYKKLQDQRIGLSVIQRNIRKWLVLRNWQWWKLYAKVKPLLSIARQEEEMKEQLKQMDKMKEDLAKTERIKKELEEQNVTLLEQKNDLFLQLQTIEDSMGDQEERVEKLIMQKADFESQIKELEERLLDEEDAAADLEGIKKKMETDNSNLKKDIGDLENTLQKAEQDKAHKDNQISTLQGEMAQQDEHIGKLNKEKKALEEANKKTSESLQAEEDKCNHLNKLKAKLEQALDELEDNLEREKKVRGDVEKAKRKVEQDLKSTQENVEDLERVKRELEENVRRKEAEISTLNSKLEDEQNLVSQLQRKVKELQARIEELEEELEAERNARAKVEKQRAELNRELEELGERLDEAGGATSAQIELNKKREAELLKIRRDLEEASLQHEAQISALRKKHQDAANEMADQVDQLQKVKSKLEKDKKDIKREMDDLESQMTHNMKNKGCSEKVMKQFESQMSDLNARLEDSQRSINELQSQKSRLQAENSDLSRQLEDAEHRVSVLSKEKSQLGSQLEDARRSLEDETRARSKLQNEVRNMHADMDAVREQLEEEQESKSDVQRQLSKANNEIQQWRSKFESEGANRTEELEDQKRKILGKLSEAEQTTEAANSKCSALEKAKSRLQQELEDMSIEVDRANANVNQMEKKQRAFDKTTAEWQSKVNSLQSELENAQKESRGYSAELYRIKASVEEYQDSIGSLRRENKNLADEIHDLTDQLSEGGRSTHELDKARRRLEMEKEELQAALEEAEGALEQEEAKVMRAQLEIATVRNEIDKRIQEKEEEFDNTRRNHQRALESMQASLEAEAKGKADAMRIKKKLEQDINELEVALDASNRGKAEMEKTVKRYQQQIREMQTSIEEEQRQRDEARESYNMAERRCTLMSGEVEELRAALEQAERARKASDNELADANDRVNELTSQVSSVQGQKRKLEGDINAMQTDLDEMHGELKGADERCKKAMADAARLADELRAEQDHSSQVEKVRKNLESQVKEFQIRLDEAEASSLKGGKKMIQKLESRVHELEAELDNEQRRHAETQKNMRKADRRLKELAFQADEDRKNQERLQELIDKLNAKIKTFKRQVEEAEEIAAINLAKYRKAQHELEEAEERADTADSTLQKFRAKSRSSVSVQRSSVSVSAN

(19) United States
(12) Patent Application Publication
(10) Pub. No.: US 2023/0016607 A1
(43) Pub. Date: Jan. 19, 2023

(54) A NOVEL METHOD TO MANUFACTURE SYNTHETIC MEAT

(71) Applicant: FYBERAWORKS FOODS, INC., St Paul, MN (US)

(72) Inventors: Chenfeng Lu, Minneapolis, MN (US); Wenjie Liu, Naperville, IL (US); Huan XIA, Naperville, IL (US)

(73) Assignee: FYBERAWORKS FOODS, INC., St Paul, MN (US)

(21) Appl. No.: 17/785,085

(22) PCT Filed: Jan. 4, 2021
(86) PCT No.: PCT/US2021/012092

§ 371 Date: Jun. 14, 2022

Related U.S. Application Data
(60) Provisional application No. 62/975,913, filed on Feb. 13, 2020.
(60) Provisional application No. 62/956,398, filed on Jan. 2, 2020.

(51) Int. Cl. A23L 13/40

(52) U.S. Cl. CPC A23L 13/45; A23L 13/43; A23Y 2240/00; A23Y 2220/00

(57) ABSTRACT

The present invention discloses a method to manufacture synthetic meat through recombinant expression of muscle proteins in edible biological hosts. The present invention particularly discloses to a method to manufacture synthetic meat that may overcome the issue of huge consumption of precious water, food and large rural area during the conventional practices of meat production. In addition, the method disclosed in the present invention aids in the significant reduction of animal slaughter.

The current application claims a priority to the U.S. Provisional Patent application Ser. No. 62/956,398 filed on Jan. 2, 2020 and a priority to the U.S. Provisional Patent application Ser. No. 62/975,913 filed on Feb. 13, 2020. The current application is filed on Jan. 4, 2021 while Jan. 2, 2021 was on a weekend.

### FIELD OF THE INVENTION

The present invention generally relates to a method to manufacture synthetic meat through recombinant expression of muscle proteins in edible biological hosts. The present invention particularly relates to a method to manufacture synthetic meat that may overcome the issue of huge consumption of precious water, food and large rural area during the conventional practices of meat production. In addition, the method disclosed in the present invention aids in the significant reduction of animal slaughter.

### BACKGROUND OF THE INVENTION

Meat has been consumed by humans for millennia even though raising animals for food is both resource intensive and environmentally unfriendly. In fact, animal-agriculture utilizes 77% of total agricultural land but only provides 17% of the human food supply. Furthermore, it uses nearly a third of freshwater available for agriculture; it discharges organic matters rich in nitrogen and phosphorus into the waterway causing algal blooms; it utilizes 70% of the medical antibiotics produced globally leading to resistant strains; and it frequently comes under criticism for inhumane and cruel slaughtering practices. With meat consumption expected to increase nearly 50% by 2050, existing animal agricultural practices cannot continue to meet market demands in a sustainable way. Additionally, the food supply is constantly threatened by pandemics such as avian influenza and African swine fever that wipe out the entire animal stocks on farms. One potential solution to these problems is the development of plant-based meat substitutes. Many challenges remain, however, in the widespread adoption of plant-based meats by the flexitarian consumer base partly because the taste, aroma, texture and nutrition of plant-based meat is not as satisfying as animal meat. Another potential solution to the above-mentioned impacts of animal agriculture is lab grown cultivated meat, consisting of muscle tissues grown via in-vitro cell culture and harvested for human consumption. The taste, flavor, and texture of lab grown meat is similar to animal meat, but there are many challenges facing the use of this technology including the need for expensive growth media, the slow growth of mammalian cells, and general scalability issues. Large gaps persist between plant-based and lab-grown meat, with regards to cost and consumer experience. The current invention encompasses development of novel animal-free meat substitutes utilizing either partial or the entire recombinant muscle structural proteins myosin and actin that closely mimic the texture and taste of animal meat for human or pet consumption. The value proposition of our technology lies in its simplicity and cost advantage over existing plant-based meat and cultivated meat technologies. To start, the cost of producing Fybraworks Foods' fungi-based alternative protein products will be equivalent to that of growing mushroom mycelium or yeasts. The production of synthetic meat will be manufactured from cheap feedstocks from corn sugar or agriculture residues and existing mature downstream processes. Next, our platform will combine the texture and flavor of muscle proteins with protein from mushroom mycelia, which already offers a rich meat-like taste (umami) and texture.

Animal meat is typically composed of water, muscle proteins, connective tissues, fat and hemoglobin if it is red meat. The main structural proteins are composed of myosin and actin, which are ubiquitously present in living organisms including yeasts. Myosin belongs to the motor protein family that has very diverse cellular functions in muscle and non-muscle cells. The main myosin in skeletal muscle cells belongs to the class II myosin, which is composed of two heavy chain globular head and coiled domains plus two pairs of light chain coiled rod domains.

The current facilities producing meat occupy large rural areas. Furthermore, current facilities producing meat consume huge quantity of precious water and feed. Moreover, current techniques involve the slaughtering of animals.

Therefore, there is a need for an improved method to manufacture synthetic meat that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Generally in one aspect of the present invention a method to manufacture synthetic meat is disclosed. Accordingly, the method may include a step of fermentation of yeast or bacteria.

In another aspect of the invention, the fermentation may be a metabolic process that produces a chemical change in organic substrate through the action of enzymes.

In another aspect of the invention, the yeast may include Pichia pastoris, Saccharomyces cerevisiae, Yarrowia lipolytica, etc. Further, the bacteria may include Escherichia coli, Bacillus subtilis, etc.

In another aspect of the invention, the yeast and the bacteria may grow in minimal media (containing minimum nutrients possible for colony growth) and obtain energy by converting sugar into alcohol or converting carbohydrates into lactic acid. Further, the yeast and the bacteria may be multiplied by at least an order of magnitude to grow further.

In another aspect of the invention, the method may include a step of producing structural muscle proteins. Further, the structural muscle proteins may include myosin II and actin. Usually, muscles are composed of two major protein filaments a thick filament and a thin filament. Further, the thick filament may be composed of the myosin.

In another aspect of the invention, the thin filament may be composed of the actin. Further, the structural muscle proteins (myosin and actin) are present in living organisms including yeasts and bacteria. In an instance, the Myosin 11 may be produced using the fermentation process.

In another aspect of the invention, the Myosin II may be composed of two heavy chains, two essential light chains, and two regulatory light chains (RLCs).

In another aspect of the invention, the structural muscle proteins may be associated with animal species such as, but not limited to, Gallus gallus (red junglefowl), Sus scrofa (wild boar), Bos taurus (cattle or cows), etc.

In another aspect of the invention, the method may include a step of cross-linking the structural muscle proteins into filaments for producing protein filaments by using chemical and biochemical processes.

In another aspect of the invention, the filaments may be “long chain of proteins, such as found in hair, muscle, or in flagella”. Further, the filaments may be often bundled together for strength and rigidity.

In another aspect of the invention, the method may include a step of blending the protein filaments with collagens, water and fats to produce synthetic meat. Further, the collagens provide strength and support to the protein filaments.

In another aspect of the invention, the blending of the protein filaments may be achieved by using a formulated amount of collagens, water, and fats. Further, the animal meat is typically composed of water, muscle proteins, connective tissues, fat and hemoglobin (if it is red meat).

In another aspect of the invention, the method may include a step of forming various meat products from the synthetic meat or blending the synthetic meat with plant-based proteins.

In another aspect of the invention, the meat products may include bacon, hams, hotdogs, prosciuttos, sausages, etc. Further, the plant-based proteins may be a food source of protein obtained from plants. Further, the plant-based proteins may be processed from chickpeas, peanuts, almonds, lentils, etc.

In another aspect of the invention, the meat products may be formulated into dry and wet pet foods for cats, dogs and other domestic animals. Further the meat products may also be consumed by farm animals and aquatic species.

In another aspect of the invention, the myosin and actin will be overexpressed intracellularly in mushrooms such as Cremini, Portobello and Button mushroom (Agaricus bisporus), Morel mushroom (Morchella esculenta), Shiitake mushroom (Lentinula edodes), Oyster mushroom (Pleurotus ostreatus), Enoki mushroom (Flammulina velutipes), and Porcini mushroom (Boletus edulis).

These recombinant mushrooms (spores) will be inoculated on fungal agar plates such as potato dextrose agar (PDA) or malt extract agar (MEA) to form mycelia. These mycelia will be cultured in a solid-state fermentation or submerged fermentation on the grain and other cellulose material rich media. The mycelia biomass will be harvested for formulation into meat products. That way the meat flavors from the myosin and actin will be combined with the meat like texture from mushrooms to simulate the meat sensory effect.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE FIGURE(S)

FIGS. 1 (a), (b) and (c) represent non-breaded nugget gel, which is squeezed at (left) minimum force; (middle) intermediate force; or (right) maximum force respectively according to the embodiments of the present invention;

FIG. 2 represents homogeneous gel matrix, achieved when the ratio of muscle protein/mushroom powder according to the embodiments of the present invention;

FIG. 3 represents post-cook non-breaded nugget part characters in the surface and bottom according to the embodiments of the present invention;

FIG. 4 represents non-breaded nugget with dried mushroom according to the embodiments of the present invention;

FIG. 5 represents non-breaded nugget according to the embodiments of the present invention;

FIGS. 6 (a) and (b) both represent breaded chicken nugget after frying according to the embodiments of the present invention;

FIGS. 7 (a), (b), and (c) all three figures represent formability of meat when being extruded before cooking according to the embodiments of the present invention;

FIGS. 8 (a) and (b) represent pet treat prototypes produced from muscle protein extracted from chicken, yeast, rice, and natural flavoring ingredients according to the embodiments of the present invention;

FIG. 9 represents SDS-PAGE (left) and Western blot (right), using anti-His antibody according to the embodiments of the present invention;

FIG. 10 represents SDS-PAGE (left) and Western blot (right), using anti-His antibody according to the embodiments of the present invention;

FIG. 11 represents Western blot (anti-His) analysis of the myosin coiled-coil domain according to the embodiments of the present invention;

FIG. 12 represents SDS-PAGE (left) and Western blot (right, anti-His) analysis of actin according to the embodiments of the present invention.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of a method to manufacture synthetic meat, embodiments of the present disclosure are not limited to use only in this context.

The present disclosure describes a method to manufacture synthetic meat. Further, the method may produce major meat structural proteins by fermentation, that resembles meat taste and structure without raising animals and slaughtering.

Animal meat is typically composed of water, muscle proteins, connective tissues, fat, and hemoglobin (if it is red meat). The main structural proteins are composed of myosin and actin, which are ubiquitously present in living organisms including yeasts. Myosin belongs to the motor protein family that has very diverse cellular functions in muscle and non-muscle cells. The main myosin in skeletal muscle cells belongs to the class II myosin, which is composed of two heavy chain globular head and coiled domains plus two pairs of light chain coiled rod domains.

In the present disclosure, these key structural muscle proteins including myosin II heavy chains and lights chains, and actins from animal species such as Gallus gallus, Sus scrofa and Bos taurus may be produced by fermentation using yeasts such as Pichia pastoris, Saccharomyces cerevisiae, and Yarrowia lipolytica or bacteria such as Escherichia coli and Bacillus subtilis that can grow in minimum media and multiply at least an order of magnitude faster than mammalian cells. These proteins may also be produced in a cell free system from yeast such as Pichia pastoris, Saccharomyces cerevisiae, or Yarrowia lipolytica or bacteria such as Escherichia coli or Bacillus subtilis, or insect such as Spodoptera frugiperda.

Alternatively, if the technical difficulties are too high to express myosin II, other myosin such as myosin V and VI may be expressed instead. These proteins may then be cross-linked into filaments by chemical or biochemical processes. These protein filaments may then be formulated with collagens, water and fat forming various meat products or blend with plant-based proteins.

Accordingly, the method may include a step of fermenting of yeast or bacteria. Further, the fermenting may be a metabolic process that produces a chemical change in organic substrate through the action of enzymes. Further, the yeast may include Pichia pastoris, Saccharomyces cerevisiae, Yarrowia lipolytica, etc. Further, the bacteria may include Escherichia coli, Bacillus subtilis, etc. Further, the yeast and the bacteria may grow in minimal media (containing minimum nutrients possible for colony growth) and obtain energy by converting sugar into alcohol or converting carbohydrates into lactic acid. Further, the yeast and the bacteria may be multiplied by at least an order of magnitude to grow further.

Further, the method may include a step of producing structural muscle proteins. Further, the structural muscle proteins may include myosin II and actin. Usually, muscles are composed of two major protein filaments a thick filament and a thin filament. Further, the thick filament may be composed of the myosin. Further, the thin filament may be composed of the actin. Further, the structural muscle proteins (myosin and actin) are present in living organisms including yeasts and bacteria. In an instance, the Myosin 11 may be produced using the fermentation process. Further, the Myosin II may be composed of two heavy chains, two essential light chains, and two regulatory light chains (RLCs). Further, in some embodiments, Myosin V and Myosin VI may be produced, instead of the Myosin II, because of technical difficulties in producing the Myosin II. Further, the structural muscle proteins may be associated with animal species such as, but not limited to, Gallus gallus (red junglefowl), Sus scrofa (wild boar), Bos Taurus (cattle or cows), etc.

Further, the method may include a step of cross-linking the structural muscle proteins into filaments for producing protein filaments by using chemical and biochemical processes. Further, the filaments may be “long chain of proteins, such as found in hair, muscle, or in flagella”. Further, the filaments may be often bundled together for strength and rigidity.

Further, the method may include a step of blending the protein filaments with collagens, water, and fats to produce synthetic meat. Further, the collagens provide strength and support to the protein filaments. Further, the blending of the protein filaments may be achieved by using a formulated amount of collagens, water and fats. Further, the animal meat is typically composed of water, muscle proteins, connective tissues, fat and hemoglobin (if it is red meat).

Further, the method may include a step of forming various meat products from the synthetic meat or blending the synthetic meat with plant-based proteins. Further, the meat products may include bacon, hams, hotdogs, prosciuttos, sausages, etc. Further, the plant-based proteins may be a food source of protein obtained from plants. Further, the plant-based proteins may be processed from chickpeas, peanuts, almonds, lentils, etc.

Further, omega-3 fatty acids may be added to the synthetic meat as a health bonus. Further, the omega-3 fatty acids may help in preventing and managing heart disease. Further, the addition of the iron-carrying protein, myoglobin to synthetic meat may produce the desired texture. Further, the addition of myoglobin or hemoglobin may change the synthetic meat's color, making it look more like conventional meat. Further, the synthetic meat product may be similar in appearance, taste, smell, texture, or other factors, with conventionally produced meat.

Further, the lack of bones and/or blood may make many conventional meat preparations, such as buffalo wings, more palatable to small children.

In regard to the synthetic meat, strict environmental controls and tissue monitoring can prevent infection of meat cultures from the outset, and any potential infection can be detected before shipment to consumers. In addition to the prevention and lack of diseases, and lack of the use of antibiotics or any other chemical substances, the synthetic meat may also leverage numerous biotechnology advancements, including increased nutrient fortification, individually-customized cellular and molecular compositions, and optimal nutritional profiles, all making it much healthier than conventional meat.

The overexpression of functional recombinant muscle myosin has been reported to be challenging owing to its large size and the complex cellular machinery required for proper myosin folding. Thus far, functional myosin expression has only been reported in murine C2C12 cell culture myoblasts and Drosophila melanogaster, but never in lower eukaryotes or microbial hosts. Myosin is assembled from two large heavy chains and two pairs of light chains, which are arranged into an asymmetrical molecule with two hydrophobic globular heads attached to a long helical rod-like hydrophilic tail. The myosin heavy chain is well studied and consists of three domains: the globular head domain (subfragment-1 of heavy meromyosin) and light-chain binding neck domain (subfragment-2 of heavy meromyosin) that binds actin and generate forces from ATPase, and the tail domain (light meromyosin) that is crucial for filament assembly via a coiled-coil motif. While essential for contraction of muscle, the head domain is notoriously difficult to overexpress. The Light MeroMyosin (LMM) displays a pattern of 28-residue repeats composed of four heptapeptides that are interrupted by four widely spaced extra amino acids called skip residues that provide flexibility to the rod domain. The current invention circumvents the challenging problem of myosin expression by expressing the coiled-coil domain of myosin instead.

Actin is one of the most abundant cytoskeleton proteins, performs a variety of cellular functions in virtually all life forms and plays a key role in the contractile apparatus of skeletal muscle in higher eukaryotes. Globular monomeric actin can polymerize into polar α-helical filaments with a barbed- and a pointed-end under precise in vivo cellular control. It has been recombinantly expressed in several microbial hosts and can be readily polymerized under physiological salt conditions in vitro with physiological monovalent or divalent cations.

TABLE 1
Exemplary muscle protein sequences

| | | Sequence | Sequence |
|---|---|---|---|
| Gene name | Gene source | type | ID |
| BtMYH1 | Bos taurus (Bovine) | Protein | 5 |
| BtACTA1 | Bos taurus(Bovine) | Protein | 12 |
| BbMYH1 | Bos taurus(Bovine) | Protein | 6 |
| ChMYH1 | Capra hircus (Goat) | Protein | 9 |
| ApMYH1 | Anas platyrhynchos | Protein | 10 |
| | (Mallard duck) | | |
| GgMYH1 | Gallus gallus (Chicken) | DNA | 1 |
| GgACTA1 | Gallus gallus(Chicken) | DNA | 2 |
| GgACTA3 | Gallus gallus(Chicken) | DNA | 14 |
| GgMYH2 | Gallus gallus(Chicken) | Protein | 3 |
| GgACTA2 | Gallus gallus(Chicken) | Protein | 4 |
| GgMYH3 | Gallus gallus(Chicken) | DNA | 15 |
| OaMYH1 | Ovis aries (sheep) | Protein | 8 |
| OcMYH1 | Oryctolagus cuniculus | Protein | 11 |
| | (rabbit) | | |
| PmMYH1 | Pecten maximus | Protein | 16 |
| SsMYH1 | Sus scrofa (pig) | Protein | 7 |
| SsMACTA1 | Sus scrofa (pig) | Protein | 13 |

One line of product is the muscle protein enriched yeast extract. The yeast fermentation process will be stopped and subject to spontaneous cell lysis. The solubles that is rich in endogenous and muscle proteins and peptides will be separated from the cell wall debris and dried for further processing. The separation of the yeast cell wall will not only improve the protein content but the digestibility of the product.

The other major family of host for muscle protein production either intracellularly or extracellularly include filamentous fungi and mushrooms. These filamentous fungi include edible species such as Fusarium venenatum, Aspergillus oryzae, Monascus purpureus, Rhizopus oryzae, Neurospora intermedia, Trichoderma sp. These mushrooms include Cremini, Portobello and Button mushroom (Agaricus bisporus), Morel mushroom (Morchella esculenta). Shiitake mushroom (Lentinula edodes), Oyster mushroom (Pleurotus ostreatus), Enoki mushroom (Flammulina velutipes), and Porcini mushroom (Boletus edulis). These recombinant mushrooms (spores) will be inoculated on fungal agar plates such as potato dextrose agar (PDA) or malt extract agar (MEA) to form mycelia. These mycelia will be cultured in a solid state fermentation or submerged fermentation on the grain and other cellulose material rich media. The mycelia biomass will be harvested for formulation into meat products. That way the meat flavors from the myosin and actin will be combined with the meat like texture from mushrooms to simulate the meat sensory effect.

To improve the texture and taste of the meat alternative, the muscle proteins expressed can be further crosslinked into filaments by chemical or biochemical processes including both co-expression of the transglutaminase and lysine monooxygenase as well as in-vitro crosslinking using the above enzymes. These processes include the additional following approaches and the combinations thereof:
1) pH adjustment and preheat treatment that unfolds tertiary structure and impacts charges of protein domains to generate the repulsion forces to keep the gel network in an expanded state.
2) High pressurization process (HPP), which has been widely used to modify protein structure through denaturation, aggregation or gelation. HPP conditions such as pressure intensity, pressurizing gradient, duration time, temperature, etc., will be optimized for interaction between animal protein and mycoprotein.
3) Increasing disulfide bond level by protein oxidation, where protein can be incubated in hydroxyl-radical-generating system.
4) Methylation on the nitrogen-containing side chain of arginine and lysine, or hydroxylation on hydroxyproline residue modified non-muscle proteins. The carbonyl content and the surface hydrophobicity will be significantly changed, which is critical for the properties of gelling and emulsification.
5) Dry or wet extrusion technology, which is commercially available and widely utilized in pulse proteins. It can convert non-gelling protein to a product with meaty texture.
6) Hydrocolloids such as sodium alginate which can gel with calcium, konjac glucomannan that is gelled with alkali, and xanthan gum/natural starch which can improve emulsion stability and binding strength.

These muscle proteins containing mycelia or mushroom will then be formulated with collagens, water and fat and/or be blended with plant based proteins, to form various meat products such as hot dog, jerky, meatball, nuggets, sausage, bacon, salami, etc.

Alternatively muscle proteins containing yeasts or yeast extract can be formulated into pet foods. The present invention provides a novel method to simulate the composition, functionality, and nutritional values of preparing Fybraworks' protein ingredients (FPI) for pet food application. The invented method involves protein textural simulation with chicken myofibrillar protein and/or hydrolyzed pea protein fortified with L-Methionine and L-Histidine that has similar amino acid composition to that of muscle protein. The protein content of Fybraworks' ingredients can be as high as 50% on a weight basis, which can be formulated into nutritionally competent pet foods for many different categories, including pets at different growing stages, main meal or treat applications for both dogs and cats (see Table 2).

Fybraworks' protein ingredients can be composed of both whole muscle protein or partial protein and peptides, which have the same nutritional values as the whole protein but with the added benefits of better digestibility and can meet the requirement of hypoallergenic grade/veterinarian prescribed foods.

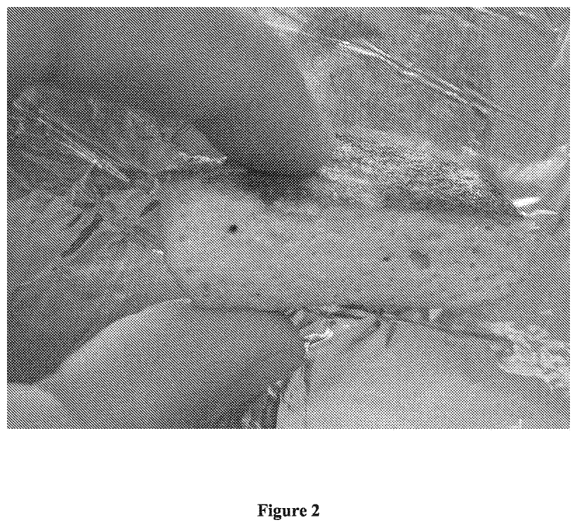

Fybraworks' protein ingredients have a distinguished savory taste throughout pet food matrix. The traditional practice to improve palatability of pet food products is through the addition of animal digest or palatant either by coating the surface of dry pet foods or mixing into the juice part of wet pet foods. The palatability distribution is typically not homogenous in these pet foods. The palatability of Fybraworks' protein ingredients is enhanced by the pleasant meaty flavors generated from Maillard reactions between peptides, amino acids, and sugar, as well as the umami taste from yeasts. The sensory tests from prototypes (FIG. 2) have confirmed the creation of a unique and homogenous savory meaty taste that's not present in pet foods with local palatant application.

TABLE 2
Examples of novel cruelty-free pet food formula range with complete
nutrition, exceptionally high digestibility and palatability

| | | | | | | Functional |
|---|---|---|---|---|---|---|
| | | | | | | premix |
| Pet food | AAFCO* | Fybraworks | | Vegetable | Flavoring | (vitamins, |
| category | Requirements | protein | Carbohydrate | oil/glycerin | ingredients | calcium, etc.) |
| Dry Dog | 22.5% protein, | 50% | 30% | 17% | 2% | 1% |
| Main Meal | 8.5% fat | | | | | |
| Dry Cat | 30% protein, | 65% | 15% | 17% | 2% | 1% |
| Main Meal | 9% fat | | | | | |
| Dog or | None | 20% | 60% | 17% | 2% | 1% |
| Cat Treats | | | | | | |

*AAFCO: The Association of American Feed Control Officials

### EXAMPLES

The below examples are only a way to illustrate some aspects of the invention, it should not construed to limit the scope of the invention.

### Example 1 Myofibrillar Protein Extraction

Muscle myofibrillar (washed myofibrils) protein was prepared in the lab from fresh chicken breasts. Visible connective tissue and fat were removed prior to chopping in a Stephan mixer-cutter. This chopped meat was combined with three parts water, stirred, and fed through a screw-fed strainer (Bibun Machine, Hiroshima, Japan) with 2.5 mm diameter mesh. Excess water was then separated from the myofibril extract each time with organza cloth. All but the straining step was repeated two more times, but the last water wash contained 0.5% (w/v) NaCl to facilitate dewatering. By the end of process, protein content of 20% w/w was achieved in this washed myofibril-rich fraction, which has been used in all the following product development (PD).

### Example 2 Evaluation of Muscle Protein to Mushroom Ratio in the “Chicken” Nugget Made from Muscle Protein and Mushroom Powder. (Sample Preparation and Cooking Procedure were Kept Constant)

“Chicken” nuggets were made of myofibrillar protein, mushroom powder, seasoning, coconut oil, water, sodium phosphate. Different ratios of myofibrillar protein (dry base)/mushroom powder have been tested at 3:1 (control), 1.5:1 (T1), 1:5 (T2), respectively. Marinade were made in a bowl by first mixing phosphate with water for 2 mins, then adding salt, seasoning and mixing for another 2 minutes. The resulting paste was chilled to 34° F. in freezer and transferred to molding equipment to form a single serving size of nugget. Lastly, nuggets go through the step of pre-dust, battering, and breading. They will be pre-fried for 30 second at 365° F. and then fully cooked in an oven until the center of product temperature reaches 165° F. Product evaluation will be conducted after overnight storage in a chiller.

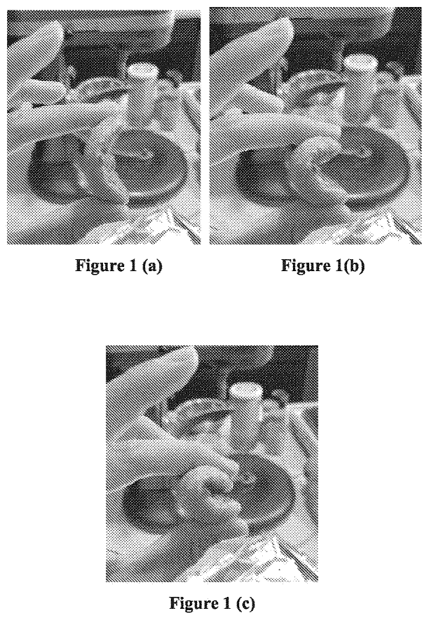

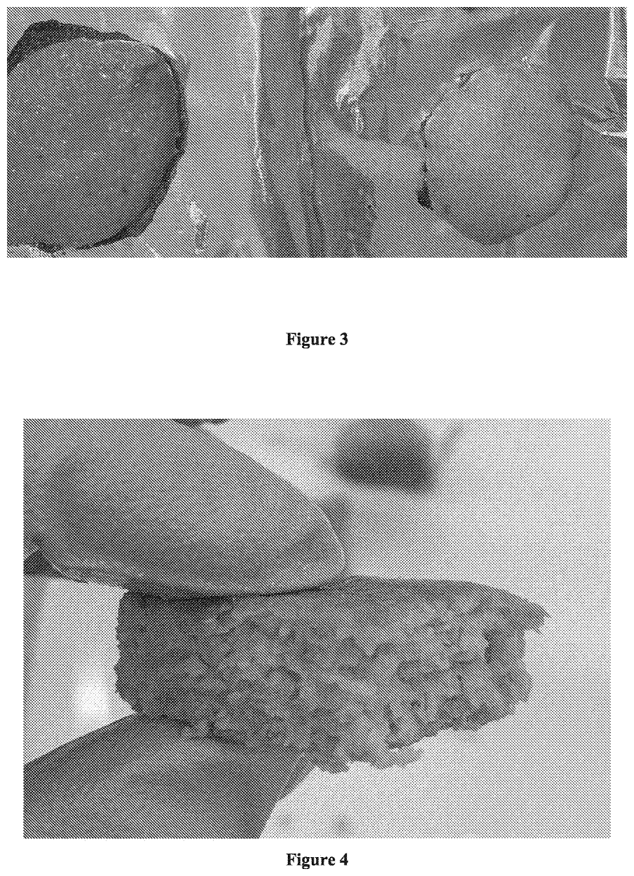

An optimized ratio of muscle protein/mushroom powder (1.5:1) was identified. Both control and T1 demonstrated elastic texture in the non-breaded part, as displayed in FIG. 1. The cross-section demonstrated homogeneous and springy gel characteristics (see FIG. 2). Once the ratio is below 1.5:1, the texture became less chewy/elastic and more brittle. Also, a large cook loss is observed in T2, as it is shown in FIG. 3 where the left one represents T2 (1:5=muscle protein:mushroom powder) and the right one represents T1 (1.5:1=muscle protein:mushroom powder). Apparently, oil overflew onto the surface of T2 sample after cooking and more overcooked debris was found on the bottom of each non-breaded meat part. Besides, T2 sample appeared burnt because there was more mushroom powder in the formula than that of T1 and control.

The results from this set of experiments implied that the ratio of muscle protein/mushroom powder are important to the texture of finished goods, which will guide our product and set protein content goals for fermentation protein content.

### Example 3 Evaluation of the Impact of Mushroom Particle Size and Methylcellulose Level to the Cooked Product. (Formulation, Sample Preparation and Cooking Procedure were Kept Constant)

The, “chicken” nuggets prototypes were made with the same procedure as described in the previous section. The variables that varies in this example were: 1) Methylcellulose was introduced into the formula to further improve texture at a low protein content product (the ratio of muscle protein/dried mushroom is increased up to 1:9 w/w). It was mixed with water at high speed first before the addition of other ingredients. 2) Different particle size of dried mushroom was evaluated for its impact on the product texture.

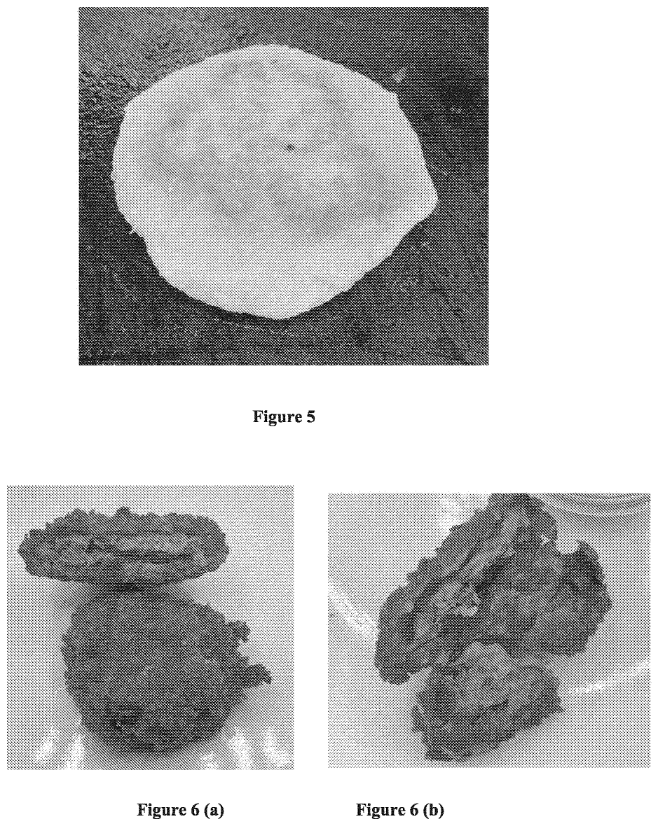

Larger particle size (˜0.5-1 cm) in dried mushroom was much preferred because it generated better texture (see FIG. 4) with a strong bite and springiness whereas the smaller particle size (˜0.1-0.5 cm) generated a brittle gel despite the homogeneous appearance. Both sizes of the mushroom powder demonstrated a great formability when cooking, as shown in FIG. 5.

These findings will help improve product texture and appearance for meat alternative products to meet different needs from customers.

### Example 4 Evaluation of the Impact of the Muscle Protein on Product Texture and Functionality

The “chicken” nuggets prototypes were made with the same procedure as described in the previous section. Effect of muscle protein on product performance was evaluated by including a no-muscle-protein control that was replaced with an equal amount of pea protein. The test formula is mimicking some off-the-shelf product except for the fact that dried mushroom with large particles (see previous section) as an alternative to TVP (i.e soy-based) is utilized in the test. The main goal is to determine if myofibrillar protein will impact product attributes in terms of appearance, texture, and breading cohesiveness.

As a conclusion, the product that contains 9% muscle protein after frying demonstrated formability, stronger bit and denser structure than the control sample despite the fact that both samples displayed similar gel structure in non-breaded part, as shown in FIG. 6. It was also observed that the product displayed slightly better bread cohesiveness than the control.

These results demonstrated advantage of a muscle myofibril protein containing had superior performance than the control without muscle proteins.

Last but not the least, it was found that at least ⅓ of salt can be removed from the original formula because the synergistic flavor boosting umami flavor between the mushroom and myofibrillar proteins. This demonstrated the additional benefit of the potential sodium reduction functions when combining mushroom and myofibrillar protein in food products.

Example 5 Ground Jerky Made from Muscle Protein and Oyster Mushroom Powder Ground jerky were made of myofibrillar protein, mushroom powder, teriyaki seasoning, cure, sodium phosphate, coconut oil, caramel and water. Different ratios of dry based myofibrillar protein over mushroom powder have been tested at 1.5:1 2:1, 2.5:1, 3:1, 3.5:1, respectively. First, myofibrillar protein was blended with cure and sodium phosphate in food processor at high shear speed for 1 minute. Then water and oil were added to mixer and blended for another 1 minute. Next both seasoning and mushroom powder were added into the mixture and blended at high shear for another 30 seconds. The final batter was extruded through jerky gun to form strips after overnight curing in a chiller. Lastly, strips will be fully cooked in a commercial dehydrator at 165° F. for 6 hours. After being removed from dehydrator products will be vacuum-packed for sensory evaluation.

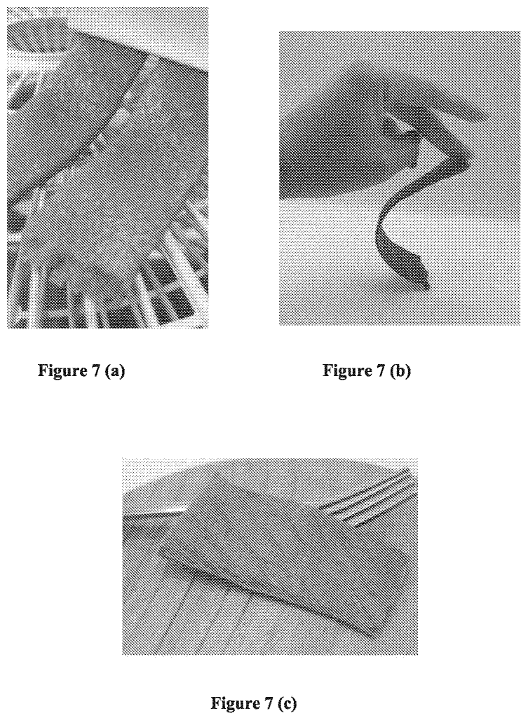

A ratio of at least 2:1 (w/w on a dry basis) of muscle protein/mushroom powder was found to be critical to achieve good formability (i.e. no breakage) (see FIG. 7). A ratio of at least 2.5:1 was found to be critical to achieve good dried meaty texture in terms of tenderness and chewiness (see FIG. 7). During dehydration procedure, the product also demonstrated real meat characters such as gelling and shrinking property, as shown in FIG. 9, which is attributed to protein denaturation and aggregation.

These findings will help improve product texture and appearance for meat alternative products to meet different needs from customers.

### Example 6 Formulation of Muscle Protein into Pet Food

Either myofibrillar protein extracted or hydrolyzed pea protein fortified with L-methionine and L-histidine was mixed with baker's yeast to simulate the yeast fermentation material at selected dry weight basis ratios. Preferred dry weight basis ratio of proteinaceous material and yeast was selected from about 5:95, to 6:94, 7:93, 8:92, 9:91, 10:90, 11:89, 12:88, 13:87, 14:86, 15:85, to 16:84, 17:83, 18:82, 19:81, or 20:80.

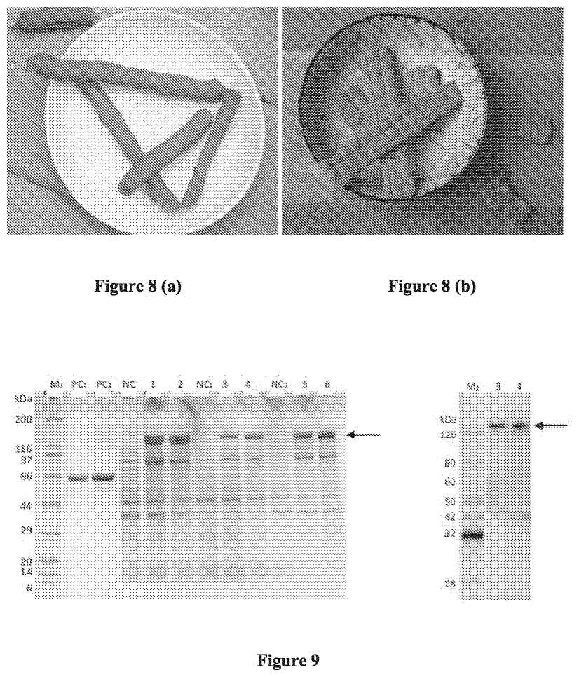

The present invention provides novel application methods to produce various pet foods using FPI. FPI was first hydrated thoroughly to encourage ionic interactions, hydrogen bonding interactions, van der Walls interactions, and hydrophobic interactions. Then vegetable oil and glycerin were added, together with flavoring and coloring ingredients. The mixture was then mixed thoroughly to yield a slurry. Carbohydrate ingredients were slowly added to form a homogeneous mixture. The material was formed into desirable shapes, heated at the temperature range of 68° C. to 95° C., with continuous ventilation, for a range of 1, to 9 hours. The pet food product was cooled and packaged to minimize moisture and aroma migration (see FIG. 8).

Preferred vegetable oils were selected from, but not limited to canola oil, sunflower seed oil, peanut oil, soybean oil, rapeseed oil, olive oil, cottonseed oil, coconut oil, corn oil, palm oil, safflower oil, sesame oil, almond oil, cashew oil, hazelnut oil, walnut oil.

Preferred flavoring and coloring ingredients were selected from, but not limited to salt, various smoke and natural flavors, molasses, dextrose, sucrose, caramel color, sodium phosphates, sodium triphosphates, sodium alginate, calcium sulfate, phosphoric acid, rosemary extract, mixed tocopherols.

Preferred carbohydrate ingredients were selected from, but not limited to rice starch, rice flour, potato starch, potato flour, tapioca starch, tapioca flour, pea starch, pea flour, corn starch, corn flour, oat flour, flaxseed flour, wheat starch, wheat flour.

Preferred forms of mixing process were selected from pumping, blending, pre-conditioning, and homogenization.

Preferred forms of heating process were selected from baking, dehydration, extrusion, and retorting.

It is envisioned that the method for producing pet foods using FPI may be carried out via a continuous, batch-wise, or a combination of both types of processing.

### Example 7 Expression of Muscle Myosin Coiled-Coil Domain in E. coli

TABLE 3
Strains and Plasmids used

| | Description | Parent |
|---|---|---|
| pFW | Expression vector harboring DNA SEQ ID | pET-30a(+) |
| F1 | NO 1 that encodes myosin coiled-coil | |
| | domain SEQ ID NO 3 | |
| pFW | Expression vector pET-30a(+) harboring | pET-30a(+) |
| F2 | DNA SEQ ID NO 2 that encodes muscle | |
| | actin coiled-coil domain SEQ ID NO 4 | |
| pFW | Integration vector harboring myosin coiled- | pPICZalphaA |
| F3 | coil domain sequence fused with a α-factor | |
| | signal peptide and 6XHis tag (SEQ ID NO | |
| | 15) under the control of the AOX promoter | |
| pFW | Integration vector harboring actin encoding | pPICZalphaA |
| F4 | sequence fused with a α-factor signal | |
| | peptide and 6XHis tag (SEQ ID NO 14) | |
| | under the control of the AOX promoter | |
| sFW | E. coli strain that expresses chicken muscle | P. pastoris X-33 |
| F1 | meromyosin | |
| sFW | E. coli strain that expresses chicken muscle | P. pastoris X-33 |
| F2 | actin | |
| sFW | P. pastoris strain that expresses chicken | P. pastoris X-33 |
| F3 | muscle meromyosin extracellularly | |
| sFW | P. pastoris strain that expresses chicken | P. pastoris X-33 |
| F4 | muscle actin extracellularly | |

Expression vector pET-30a(+) harboring DNA SEQ ID NO 1 that encodes myosin coiled-coil domain SEQ ID NO 3 were transformed into E. coli BL21 Star™ (DE3) competent cells. A single colony was inoculated into LB medium containing kanamycin; cultures were incubated in 37° C. at 200 rpm. Once cell density reached to OD=0.6-0.8 at 600 nm, 0.5 mM IPTG was introduced for induction. SDS-PAGE and Western blot were used to monitor the expression (FIG. 9). The expression level reached 40 mg/L and solubility of 40%.

### Example 8 Expression of Muscle Actin Coiled-Coil Domain in E. coli

Expression vector pET-30a(+) harboring DNA sequence SEQ ID NO 2 that encodes muscle actin coiled-coil domain SEQ ID NO 4 were transformed into E. coli BL21 Star™ (DE3) competent cells.

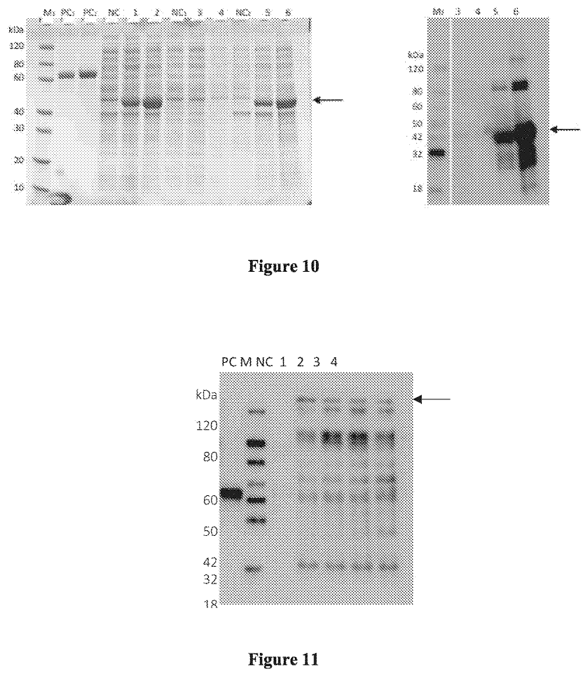

The expression level of muscle actin coiled-coil domain reached 35 mg/L and little solubility. The lack of solubility for actin is likely due to the spontaneous polymerization, other than the 43 kDa monomer, 86 kDa dimer and 129 kDa trimer are also visible from the western blot (see FIG. 10).

### Example 9 Expression of Muscle Myosin Coiled-Coil Domain in P. pastoris

Myosin coiled-coil domain sequence fused with a α-factor signal peptide and 6×His tag (SEQ ID NO 15) was cloned into an integration vector pPICZalphaA under the control of the AOX promoter, transformed into P. pastoris X-33 expression strains, grown on BMGY/BMMY and the expression will be induced by the addition of methanol for 96 h at 28° C. at 200 rpm. The expression level is low but detectable in the cell pellet (see FIG. 1l).

### Example 10 Expression of Muscle Actin Coiled-Coil Domain in P. pastoris

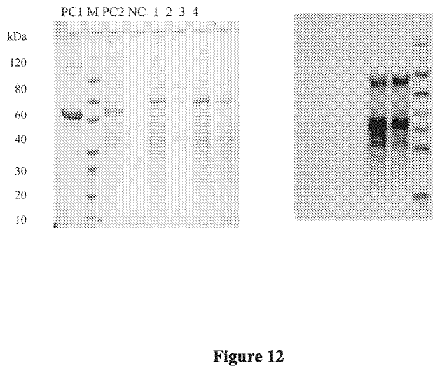

Actin encoding sequence fused with a α-factor signal peptide and 6×His tag (SEQ ID NO 14) was cloned into an integration vector pPICZalphaA under the control of the AOX promoter, transformed into P. pastoris X-33 expression strains, grown on BMGY/BMMY and the expression will be induced by the addition of methanol for 96 h at 28° C. at 200 rpm. Clone #6 has the highest level of expression (see FIG. 12) and also prone to spontaneous polymerization that is similar to its expression in E. coli.

The synthetic meat has several prospective health, environmental, cultural, and economic considerations in comparison to conventional meat.

Although the invention has been explained in relation to its preferred embodiment, it is to be understood that many other possible modifications and variations can be made without departing from the spirit and scope of the invention.

What is claimed is:

1. A novel method for producing synthetic meat through recombinant expression of muscle proteins in edible biological hosts comprises the following steps:
a) inoculating recombinant source material into a growth medium;
b) culturing the above mentioned recombinant source material in a fermentation process in the presence of yeast or bacteria or other edible biological hosts on minimal growth medium to form structural muscle protein;
c) propagating the yeast and bacteria for multitude growth;
d) cross-linking of the above mentioned structural muscle protein into filaments for producing protein into filaments for producing protein filament by using biochemical and chemical process;
e) bundling the above-mentioned cross linking filaments together to provide strength and rigidity;
f) blending the above obtained protein filament with collagens, water and fats to produce desired synthetic meat;
wherein, the resultant animal meat may be characterized to be composed of collagens, water, muscle proteins, connective tissues fat and heamoglobin and may be further blended with plant based proteins in addition to fortification with other essential nutrients.

2. The novel method as claimed in claim 1, wherein, the edible biological hosts may be selected from the group consisting of filamentous fungi include edible species such as Fusarium venenatum, Aspergillus oryzae, Monascus purpureus, Rhizopus oryzae, Neurospora intermedia, Trichoderma sp. These mushrooms include Cremini, Portobello and Button mushroom (Agaricus bisporus), Morel mushroom (Morchella esculenta), Shiitake mushroom (Lentinula edodes), Oyster mushroom (Pleurotus ostreatus), Enoki mushroom (Flammulina velutipes), and Porcini mushroom (Boletus edulis)

3. The novel method as claimed in claim 1, wherein the cell comprises the nucleic acid encoding muscle protein containing polypeptide is selected from the group consisting of actins, myosins, truncated actins, truncated myosins and a combination thereof.

4. The novel method as claimed in claim 1, wherein the cell comprises the nucleic acid encoding the truncated muscle myosin is a coiled-coil.

5. The novel method as claimed in claim 1, wherein the cell comprises the promoter sequence is a constitutive promoter.

6. The novel method as claimed in claim 1, wherein the cell comprises the promoter sequence is a tissue-specific promoter.

7. The novel method as claimed in claim 1, wherein the cell comprises the promoter sequence is an inducible promoter.

8. The novel method as claimed in claim 1, wherein the cell further comprising of a transcription termination region.

9. The novel method as claimed in claim 1, wherein the cell comprises the signal peptide is a secretion signal peptide.

10. The novel method as claimed in claim 1 wherein, the yeast may be selected from Saccharomyces cerevisiae, Pichia sp. including Pichia pastoris, Candida sp., Komagataella sp., Kluyveromyces lactis, Yarrowia lipolytica, Issatchenkia sp., or a mixture thereof.

11. The novel method as claimed in claim 1 wherein, the bacteria may be selected from Streptococcus, Lactobacillus, Bacillus, Escherichia, Salmonella, Neisseria, Acetobactor, Arthrobacter, Aspergillus, Bifdobacterium, Corynebacterium, Pseudomonas fluorescens or a mixture thereof.

12. The novel method as claimed in claim 1 wherein, the structural muscle protein produced from the group of myosin II and muscle actins from animal species such as duck (Anas sp.), rabbit (Oryctolagus sp.), goose (Anser sp.), bison and buffalo (Bos bison, Bubalus sp.), sheep (Ovis sp.), goat (Capra sp.), horse (Equus sp.), fish (Oncorhynchus sp., Oreochromus sp., Gadus sp., Salvelinus sp., Morone sp., Bidyanus sp., Silurus sp., Seriola sp., Perca sp.,), scallop (Pectinida sp.), shrimp (Litopenaeus sp., Penaeus sp., Acetes sp., Trachysalambria sp., Fenneropenaeus sp., Pandalus borealis), crab (Carcinus sp., Callinectes sapidus, Metacarcinus magister, Chionoecetes opilio), oyster and mussel (Pteriomorphia sp.), chicken (Gallus gallus), pork (Sus scrofa) and beef (Bos taurus)

13. The novel method as claimed in claim 1 wherein, the filament produced may be from group of “long chain of proteins, such as found in hair, muscle, or in flagella.

14. The novel method as claimed in claim 1 wherein, the blending of plant based proteins may be selected from chickpeas, peanuts, almonds, lentils, etc.

15. The novel method as claimed in claim 1 wherein, the resultant animal meat may be fortified with group of nutrients selected from omega-3 fatty acids to increase its nutritional value.

16. The novel method as claimed in claim 1 wherein, the resultant animal meat can be applied to bacon, hams, hotdogs, prosciuttos, sausages, pet food, animal feed, etc. Further, the plant-based proteins may be a food source of protein obtained from plants.

17. The novel method as claimed in claim 1 wherein, the resultant meat may be similar in appearance, taste, smell, texture, or other factors, with conventionally produced meat.

18. The novel method as claimed in claim 1 wherein, the animal meat prospective health, environmental, cultural, and economic considerations in comparison to conventional meat.

19. The novel method as claimed in claim 1 wherein, the improved method aids in significant reduction of animals of slaughter.
